(19) United States
(12) Patent Application Publication
(10) Pub. No.: US 2012/0282683 A1
(43) Pub. Date: Nov. 8, 2012

(54) SAMPLE ANALYSIS DEVICE

(76) Inventor: Kazunori Mototsu, Kobe-shi (JP)

(21) Appl. No.: 13/549,953

(22) Filed: Jul. 16, 2012

Related U.S. Application Data
(63) Continuation of application No. PCT/JP2011/050470, filed on Jan. 13, 2011.

(30) Foreign Application Priority Data
Jan. 21, 2010 (JP) JP2010-010836

(51) Int. Cl. G01N 21/75; C12M 1/40

(52) U.S. Cl. 435/287.2; 422/82.05; 422/69

(57) ABSTRACT

Disclosed is a sample analysis device provided with: a first sample processing portion which is disposed in a first layer and performs some of a plurality of processes on a sample in a container; a second sample processing portion which is disposed in a second layer located above or under the first layer and performs at least some other processes among the plurality of processes on the sample in the container, the some of the plurality of processes having been performed on the sample; and a container transfer portion which transfers the container, which contains the sample on which the some of the processes have been performed, from the first layer to the second layer.

### RELATED APPLICATIONS

This application is a continuation of PCT/JP2011/050470 filed on Jan. 13, 2011, which claims priority to Japanese Application No. 2010-010836 filed on Jan. 21, 2010. The entire contents of these applications are incorporated herein by reference.

### BACKGROUND OF THE INVENTION

1. Field of the Invention

The present invention relates to a sample analysis device, and more particularly, it relates to a sample analysis device analyzing a sample by carrying out a plurality of processes.

2. Description of the Related Art

A sample analysis device that analyzes a sample by carrying out a plurality of processes is known in general (refer to Japanese Patent Laying-Open No. 10-62433, for example).

In the aforementioned Japanese Patent Laying-Open No. 10-62433, there is disclosed an automatic immunoassay device including a cartridge storing portion that stores a cartridge for storing a sample and reagents, a reaction line that successively transfers the cartridge to various operating positions while keeping the same at a prescribed reaction temperature, a sample injection device that injects the sample into the cartridge on the reaction line, a mixing mechanism for mixing various reagents such as magnetic particles, an enzyme-labeled reagent and a diluent with the sample in the cartridge on the reaction line, a washer that performs BF (Bound Free) separation of separating (removing) an unreacted labeled reagent and the sample from a specimen in which the sample and the reagents have been mixed, a measurement portion that measures the measurement specimen in the cartridge and a cartridge transportation mechanism that transfers the cartridge from the reaction line to the measurement portion. This automatic immunoassay device according to Japanese Patent Laying-Open No. 10-62433 is so formed that various processes such as injection of the sample, mixing of the reagents and the sample and the BF separation are carried out by the respective units (the sample injection device, the mixing mechanism and the washer etc.) on respective positions of the reaction line in a process in which the cartridge set on the reaction line is transferred toward an end point of the reaction line. The automatic immunoassay device is so formed that the cartridge is thereafter transferred to the measurement portion by the cartridge transportation mechanism on the end point of the reaction line while the measurement specimen in the cartridge is measured by the measurement portion.

In a device that carries out a large number of processes as the automatic immunoassay device described in the aforementioned Japanese Patent Laying-Open No. 10-62433, however, it is necessary to arrange a plurality of units that carries out the respective processes in the device in order to smoothly perform the processes, and hence there is such a problem that the size of the device so horizontally increases that a set area of the device increases.

### SUMMARY OF THE INVENTION

A first aspect of the present invention is a sample analysis device that analyzes a sample by carrying out a plurality of processes on the sample in a container and has a plurality of layers. The sample analysis device comprises: a first sample processing portion that is arranged in a first layer and that is configured to carry out one part of the plurality of processes on the sample in the container; a second sample processing portion that is arranged in a second layer positioned above or under the first layer and that is configured to carry out at least another part of the plurality of processes on the sample in the container, the one part of the plurality of processes having been carried out on the sample in the container; and a container transfer portion configured to transfer the container, which contains the sample on which the one part of the plurality of processes has been carried out, from the first layer to the second layer.

A second aspect of the present invention is a sample analysis device that analyzes a sample by carrying out a plurality of processes on the sample in a container. The sample analysis device comprises: a first base; a first sample processing portion that is arranged on the first base and that is configured to carry out one part of the plurality of processes on the sample in the container; a second base arranged above or under the first base; and a second sample processing portion that is arranged on the second base and that is configured to carry out at least another part of the plurality of processes on the sample in the container, the one part of the plurality of processes having been carried out on the sample in the container; and a container transfer portion configured to transfer the container, which contains the sample on which the one part of the plurality of processes has been carried out, from the first sample processing portion to the second sample processing portion.

### BRIEF DESCRIPTION OF THE DRAWINGS

FIG. 1 is a front surface-side perspective view showing the overall structure of an immunoanalyzer according to an embodiment of the present invention;

FIG. 2 is a back surface-side perspective view showing the overall structure of the immunoanalyzer according to the embodiment of the present invention;

FIG. 3 is a side elevational view on the back surface side of the immunoanalyzer according to the embodiment shown in FIG. 2;

FIG. 4 is a plan view showing an upper layer U of the immunoanalyzer according to the embodiment shown in FIG. 1;

FIG. 5 is a plan view showing a middle layer M of the immunoanalyzer according to the embodiment shown in FIG. 1;

FIG. 6 is a plan view showing a lower layer L of the immunoanalyzer according to the embodiment shown in FIG. 1;

FIG. 7 is a block diagram for illustrating the structure of the immunoanalyzer according to the embodiment shown in FIG. 1;

FIG. 8 is a block diagram for illustrating the structure of the immunoanalyzer according to the embodiment shown in FIG. 1;

FIG. 9 is a diagram showing a measurement flow in the immunoanalyzer according to the embodiment shown in FIG. 1;

FIG. 10 is a schematic diagram for illustrating the measurement flow in the immunoanalyzer according to the embodiment shown in FIG. 1;

FIG. 11 a schematic diagram showing reaction between an antigen in a sample measured in the immunoanalyzer according to the embodiment shown in FIG. 1 and various reagents; and

FIG. 12 is a schematic diagram showing a three-layer structure of the immunoanalyzer according to the embodiment shown in FIG. 1.

### DETAILED DESCRIPTION OF THE PREFERRED EMBODIMENTS

An embodiment embodying the present invention is now described on the basis of the drawings.

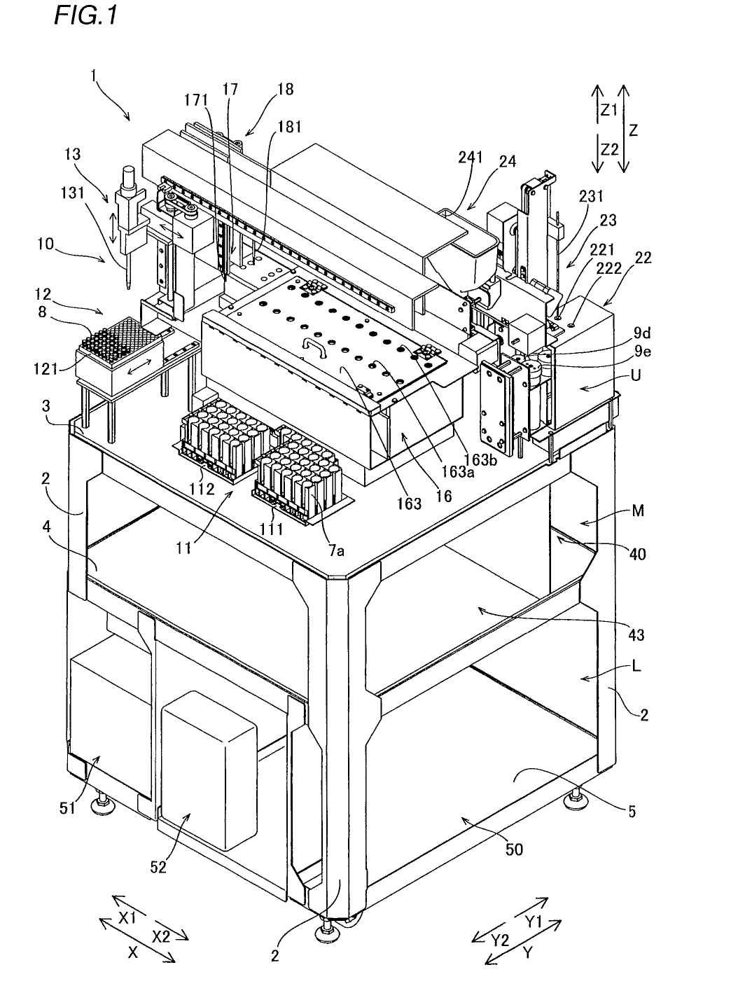

First, the overall structure of an immunoanalyzer 1 according to the embodiment of the present invention is described with reference to FIGS. 1 to 8, FIG. 10 and FIG. 12.

The immunoanalyzer 1 according to the embodiment of the present invention is a device for performing testing of various items such as protein, a tumor marker and a thyroid hormone related to an infectious disease (hepatitis B, hepatitis C or the like) with a sample such as blood.

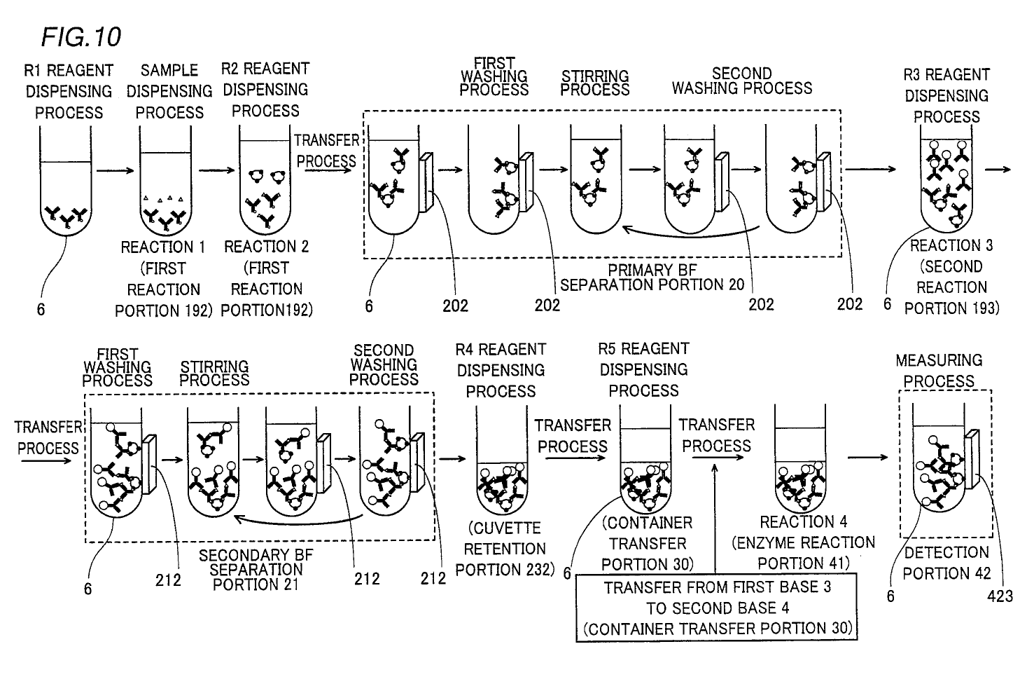

This immunoanalyzer 1 is a device that quantitatively measures or qualitatively measures an antigen, an antibody etc. contained in a sample (blood specimen) such as blood which is a measuring object. This immunoanalyzer 1 is so formed, in a case of quantitatively measuring an antigen contained in a sample, as to bind magnetic particles (R2 reagent) to a capturing antibody (R1 reagent) bound to the antigen contained in the sample and as to thereafter attract a composite of the capturing antibody and the magnetic particles to a magnet 202 (see FIG. 10) of a primary BF (Bound Free) separation portion 20 thereby removing the R1 reagent containing an unreacted (Free) capturing antibody. After binding the antigen to which the magnetic particles are bound and a labeled antibody (R3 reagent) to each other, the immunoanalyzer 1 attracts a composite of the bound (Bound) magnetic particles, the antigen and the labeled antibody to a magnet 212 (see FIG. 10) of a secondary BF separation portion 21, thereby removing the R3 reagent containing an unreacted (Free) labeled antibody. After adding a dispersion liquid (R4 reagent) and a light-emitting substrate (R5 reagent) emitting light in a reaction process with the labeled antibody, the immunoanalyzer 1 measures the quantity of light emission caused by reaction between the labeled antibody and the light-emitting substrate. Through such a plurality of processes, the immunoanalyzer 1 quantitatively measures the antigen contained in the sample bound to the labeled antibody.

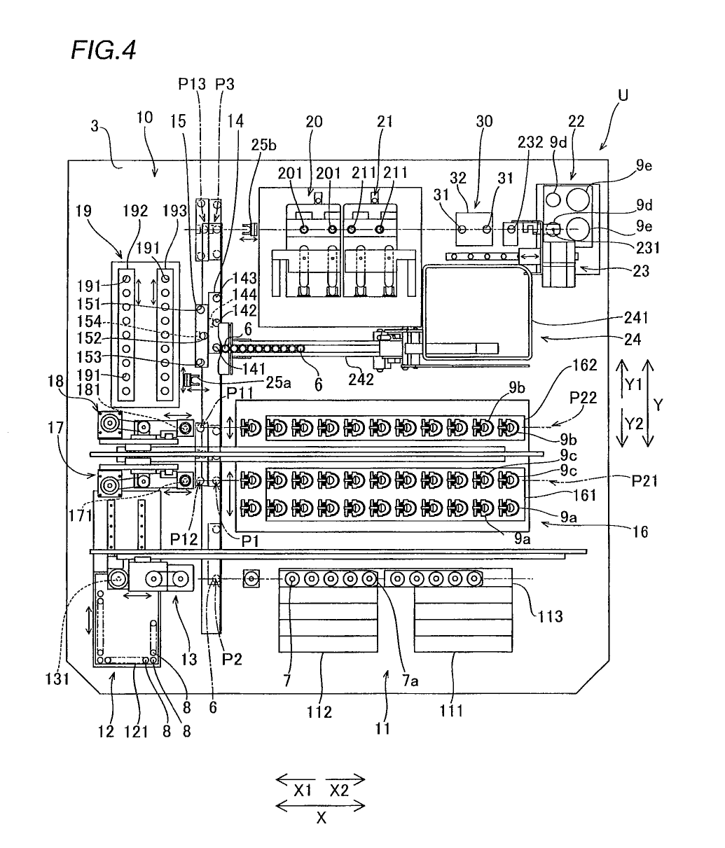

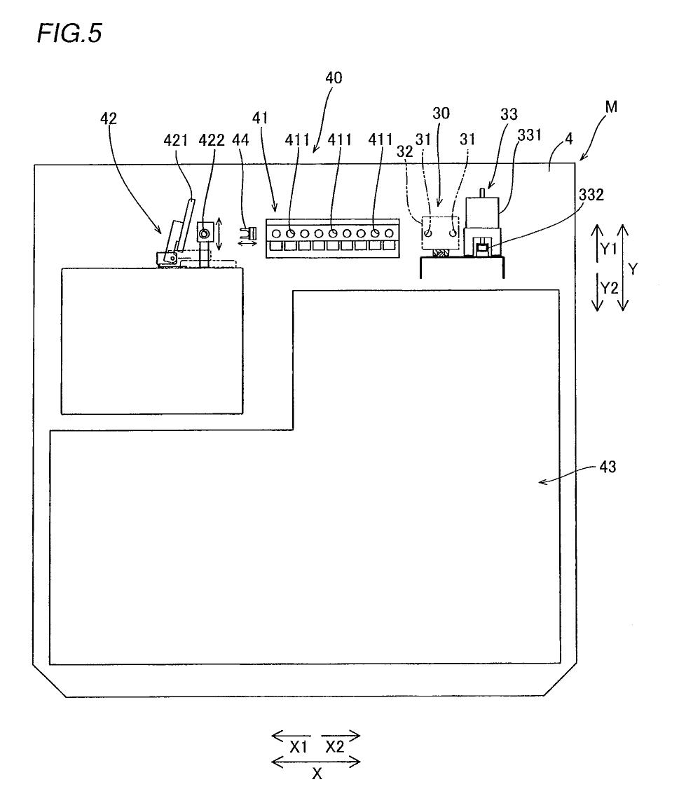

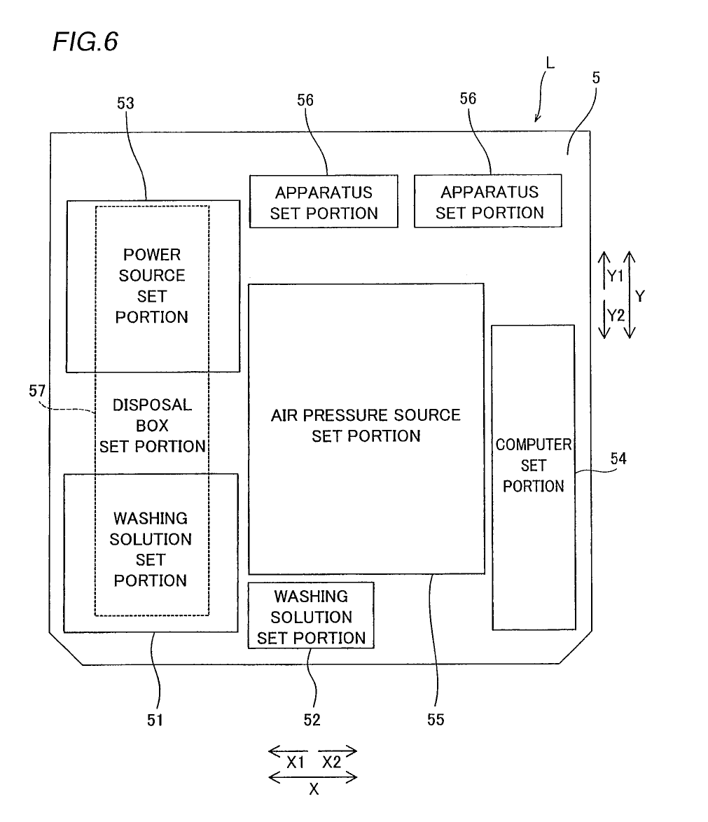

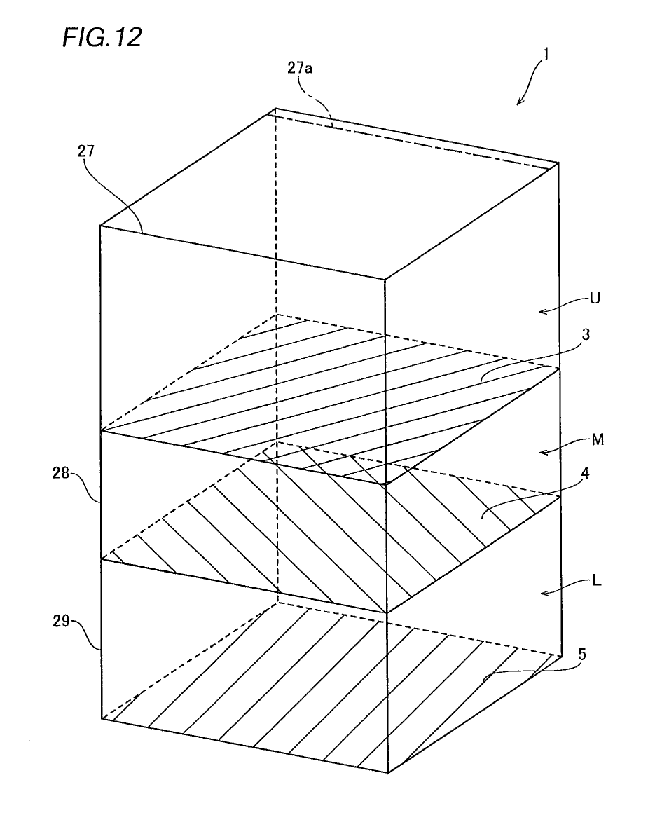

According to this embodiment, a first base 3 is arranged on the uppermost portion, a second base 4 is arranged under (arrow Z2 direction) the first base 3, and a third base 5 is arranged under the first base 3 and the second base 4 in a frame 2 of the immunoanalyzer 1, as shown in FIGS. 1 to 3. Thus, the immunoanalyzer 1 has a three-layer structure consisting of an upper layer U (first layer) located above the first base 3, a middle layer M (second layer) located between the first base 3 and the second base 4, and a lower layer L (lower set layer) located between the second base 4 and the third base 5, as shown in FIG. 12. As shown in FIGS. 1 to 3, the first base 3 (see FIG. 4), the second base 4 (see FIG. 5) and the third base 5 (see FIG. 6) have substantially square identical shapes in plan view, and are arranged to line up at prescribed intervals from each other in the vertical direction (direction Z) to completely overlap with each other in plan view. A first sample processing portion 10 is provided on the first base 3, and a second sample processing portion 40 is provided on the second base 4. Set regions such as washing solution set portions 51 and 52 (see FIG. 6) for setting washing solutions described later are provided on the third base 5 provided most downward (arrow Z2 direction).

According to this embodiment, the immunoanalyzer 1 is provided with a container transfer portion 30 for transporting cuvettes (containers) 6 from the upper layer U to the middle layer M. The cuvettes 6 are transparent containers, which are employed for storing liquids such as samples and reagents, reacting the samples and the reagents with each other, and detecting prescribed components in the stored liquids. The container transfer portion 30 is formed to transfer the cuvettes 6 from the upper layer U to the middle layer M after various processes such as a dispensation process of the reagents into the samples in the cuvettes 6 and a prescribed reaction process with respect to the liquids in the cuvettes 6 are performed in the first sample processing portion 10.

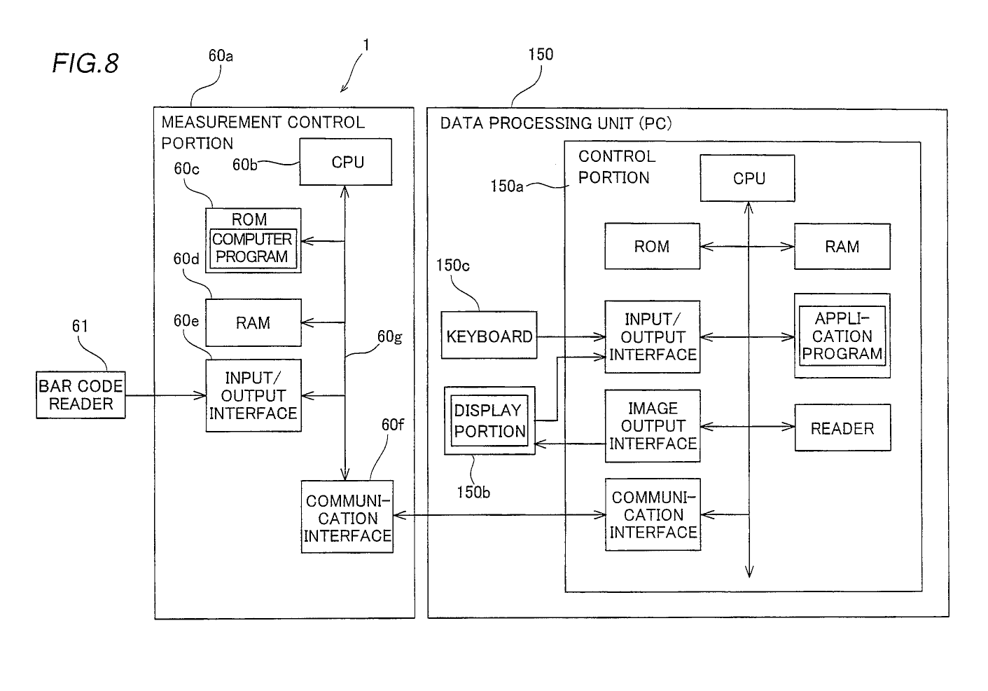

The immunoanalyzer 1 is formed to perform measurement and an analytical process of the samples with the first sample processing portion 10 and the second sample processing portion 40 having functions of performing measurement of blood forming the samples and a data processing unit (PC) 150 (see FIG. 8) that obtains analytical results by analyzing measurement results output from a detection portion 42, described later, of the second sample processing portion 40.

The first sample processing portion 10 on the first base 3 is formed to carry out part of a plurality of processes carried out by the immunoanalyzer 1 on the samples in the cuvettes 6, and mainly constituted of a sample rack set portion 11, a chip rack set portion 12, a sample dispensing arm 13, a first cuvette transport portion 14 and a second cuvette transport portion 15, a first reagent set unit 16, a first reagent dispensing arm 17, a second reagent dispensing arm 18, an antigen-antibody reaction table 19, a primary BF separation portion 20 and a secondary BF separation portion 21, a second reagent set unit 22, a third reagent dispensing arm 23 and a cuvette supply portion 24, as shown in FIG. 4.

The sample rack set portion 11 of the first sample processing portion 10 is so formed that a rack 7a on which a plurality of (five) test tubes 7 storing samples are placed can be set by the user, as shown in FIG. 4. This sample rack set portion 11 has a rack set portion 111 for setting the rack 7a on which the test tubes 7 storing unprocessed samples are placed, a rack reserving portion 112 for reserving the rack 7a on which the test tubes 7 storing samples already subjected to dispensation process are placed, and a lateral feed portion 113 for laterally feeding the rack 7a set on the rack set portion 111 in an arrow X1 direction and transferring the same to the rack reserving portion 112. The lateral feed portion 113 is so provided that the position in a direction Y coincides with the position of the sample dispensing arm 13. The sample dispensing arm 13 is formed to be movable in a direction X and a direction Z (vertical direction) as described later. The test tubes 7 storing the unprocessed samples are so transferred to prescribed positions on the lateral feed portion 113 that suction of the samples such as blood in the test tubes 7 is performed by the sample dispensing arm 13 and the rack 7a on which the test tubes 7 are placed is reserved in the rack reserving portion 112.

The chip rack set portion 12 is provided for retaining a chip rack 121 retaining a large number of pipette chips 8 (see FIG. 1) employed for suction and discharge of the samples in the form of a matrix (in the form of rows and columns). The chip rack set portion 12 is formed to be capable of moving the chip rack 121 in the direction Y. Thus, the chip rack set portion 12 is formed to move the chip rack 121 in the direction Y and to move the sample dispensing arm 13 in the direction X and the direction Z (vertical direction) so that the sample dispensing arm 13 mounts the pipette chips 8 retained on arbitrary positions of the chip rack 121.

The sample dispensing arm 13 has a function of dispensing the sample in each test tube 7 transported onto the lateral feed portion 113 of the sample rack set portion 11 into each cuvette 6 retained in a cuvette receiving hole 141, described later, of the first cuvette transport portion 14. This sample dispensing arm 13 is formed to be movable above (arrow Z1 direction, see FIG. 1) the sample rack set portion 11 (lateral feed portion 113), the chip rack set portion 12 and the first cuvette transport portion 14 in the direction X on the first base 3. Further, the sample dispensing arm 13 has a pipette portion 131 (see FIG. 1) extending downward (arrow Z2 direction), and is formed to be capable of raising/lowering this pipette portion 131 in the vertical direction (direction Z). The pipette chips 8 (see FIG. 1) retained in the chip rack 12 of the chip rack set portion 12 are mounted on the forward end of the pipette portion 131. The sample dispensing arm 13 mounts the pipette chips 8 on the pipette portion 13 above the chip rack set portion 12, moves in an arrow X2 direction up to a sucking position on the lateral feed portion 113 of the sample rack set portion 11, and sucks the samples in the test tubes 7 into the pipette portion 131. The sample dispensing arm 13 is formed to thereafter move in the arrow X1 direction from the sucking position on the lateral feed portion 113 and to dispense the sucked samples into the cuvettes 6 transported to a sample dispensation position P2, as shown in FIG. 4.

The first cuvette transport portion 14 has three cuvette receiving holes 141, 142 and 143 for retaining the cuvettes 6, and has a function of transporting the retained cuvettes 6 to prescribed positions. More specifically, the first cuvette transport portion 14 is formed to be movable in the direction Y, and formed to be capable of transporting the retained cuvettes 6 to an R1 reagent dispensing position P1, the sample dispensing position P2 and a first BF delivery position P3 etc. A magnet 144 (see a broken line in FIG. 4) is provided on a side portion of the cuvette receiving hole 142 of the first cuvette transport portion 14, and has a function of collecting magnetic particles in the cuvette 6 retained in the cuvette receiving hole 142.

The second cuvette transport portion 15 has three cuvette receiving holes 151, 152 and 153 for retaining the cuvettes 6 and a magnet 154 (see a broken line in FIG. 4) provided on a side portion of the cuvette receiving hole 152 similarly to the first cuvette transport portion 14, and has a function of transporting the retained cuvettes 6 to prescribed positions. More specifically, the second cuvette transport portion 15 is formed to be movable in the direction Y, and formed to be capable of transporting the retained cuvettes 6 to an R2 reagent dispensing position P11, an R3 reagent dispensing position P12 and a second BF delivery position P13 etc.

The first reagent set unit 16 includes an R1/R3 set portion 161 for setting reagent containers 9a in which an R1 reagent containing a capturing antibody is stored and reagent containers 9c in which an R3 reagent containing a labeled antibody is stored and an R2 set portion 162 for setting reagent containers 9b in which an R2 reagent containing magnetic particles is stored, and is so formed that these reagent containers 9a, 9b and 9c are settable and exchangeable by the user. A plurality of reagent containers 9a and the reagent containers 9c are set on the R1/R3 set portion 161 to extend in the direction X respectively. The R1/R3 set portion 161 is formed to be movable in the direction Y, and formed to be capable of arranging a column (column in the direction X) of the reagent containers 9a and a column of the reagent containers 9c on a sucking position P21 whose position in the direction Y coincides with the first reagent dispensing arm 17 respectively. The column of the reagent containers 9c containing the R3 reagent is arranged on the sucking position P21 in FIG. 4. The R2 set portion 162 is arranged on a sucking position P22 whose position in the direction Y coincides with the second reagent dispensing arm 18, while the reagent containers 9b are set in plural to extend in the direction X. The R2 set portion 162 is formed to be swingable in the direction Y, and formed to be capable of uniformly stirring the magnetic particles contained in the R2 reagent in the reagent containers 9b. As shown in FIG. 1, the first reagent set unit 16 includes a lid portion 163 having a plurality of hole portions 163a formed on positions corresponding to the sucking position P21 for the R1 reagent and the R3 reagent by the first reagent dispensing arm 17 and a plurality of hole portions 163b formed on positions corresponding to the sucking position P21 for the R2 reagent by the second reagent dispensing arm 18, and is so formed that the reagents are sucked through these hole portions 163a and 163b.

The first reagent dispensing arm 17 has a function for dispensing the reagents (the R1 reagent and the R3 reagent) in the reagent containers 9a and the reagent containers 9c set on the R1/R3 set portion 161 of the first reagent set unit 16 into the cuvettes 6. This first reagent dispensing arm 17 is formed to be movable above the first reagent set unit 16 (hole portions 163a) in the direction X, and has a pipette 171 (see FIG. 1) movable in the vertical direction (direction Z). In a state where a reagent column (column of the reagent containers 9a or the reagent containers 9c) to be subjected to dispensation is arranged on the sucking position P21 by the R1/R3 set portion 161, the first reagent dispensing arm 17 moves in the direction X and sucks the reagent from the reagent containers (the reagent containers 9a or the reagent containers 9c) to be subjected to dispensation with the pipette 171. The first reagent dispensing arm 17 is formed to be capable of dispensing the sucked R1 reagent into the cuvettes 6 transported to the reagent dispensing position P1 and dispensing the sucked R3 reagent into the cuvettes 6 transported to the R3 reagent dispensing position P12.

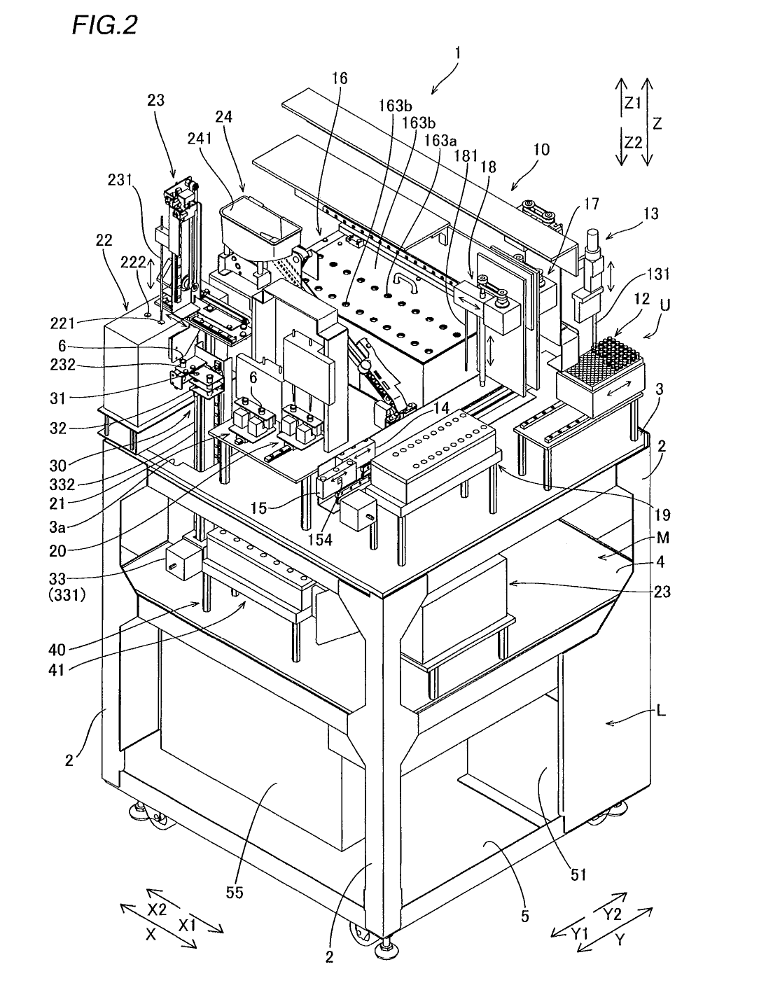

The second reagent dispensing arm 18 has a function for dispensing the reagent (R2 reagent) in the reagent containers 9b set on the R2 set portion 162 of the first reagent set unit 16 into the cuvettes 6. This second reagent dispensing arm 18 is formed to be movable above the first reagent set unit 16 (hole portions 163b) in the direction X, and has a pipette 181 (see FIG. 2) movable in the vertical direction (direction Z). The second reagent dispensing arm 18 is formed to be capable of sucking the reagent from the reagent containers 9b to be subjected to dispensation with the pipette 181 by moving in the direction X and dispensing the sucked R2 reagent into the cuvettes 6 transported to the R2 reagent dispensing position P11.

The antigen-antibody reaction table 19 has a first reaction portion 192 on which a plurality of storage holes 191 for retaining the cuvettes 6 respectively and performing incubation are provided in the form of a column extending in the direction Y and a second reaction portion 193. The first reaction portion 192 is provided for performing reaction (reaction 1) between the R1 reagent (capturing antibody) and antigens in the samples and reaction (reaction 2) binding specimens (capturing antibody to which the antigens are bound) after completion of the reaction 1 and the R2 reagent (magnetic particles) to each other. The second reaction portion 193 is provided for performing reaction (reaction 3) binding specimens (the R1 reagent, the samples and the R2 reagent), on which the reaction 1, the reaction 2 and primary BF separation have been performed, and the R3 reagent (labeled antibody) to each other. The first reaction portion 192 and the second reaction portion 193 are formed to be swingable in the direction Y respectively, and capable of stirring the R2 reagent (magnetic particles) also during incubation.

The primary BF separation portion 20 is provided for separating (primary BF separation) an unreacted R1 reagent (unnecessary components) and the magnetic particles from the specimens on which the reaction 1 and the reaction 2 with the antigen-antibody reaction table 19 have been performed. The primary BF separation portion 20 mainly has two set holes 201 for setting the cuvettes 6 containing the samples, the R1 reagent and the R2 reagent, the magnet 202 (see FIG. 10) collecting the magnetic particles, a washing mechanism (not shown) having a nozzle (not shown) performing supply of a washing solution and removal (suction) of unnecessary components, and a stirring mechanism (not shown) stirring the washing solution, the unnecessary components and the magnetic particles in the cuvettes 6. The primary BF separation portion 20 is formed to remove the unreacted R1 reagent (unnecessary component) in the cuvettes 6 through four washing processes with the aforementioned respective mechanisms, and to separate the unreacted R1 reagent (unnecessary component) and the magnetic particles.

The secondary BF separation portion 21 has a structure similar to that of the primary BF separation portion 20, and is provided for separating (secondary BF separation) an unreacted R3 reagent (unnecessary component) not bound to the antigens in the samples and the magnetic particles from the specimens on which the reaction 3 by the antigen-antibody reaction table 19 (second reaction portion 193) has been performed. The secondary BF separation portion 21 is formed to separate the unreacted R3 reagent (unnecessary component) and the magnetic particles from the specimens containing the samples, the R1 reagent, the R2 reagent and the R3 reagent in the cuvettes 6 set in set holes 211 with the magnet 212 (see FIG. 10), a washing mechanism (not shown) and a stirring mechanism (not shown).

The second reagent set unit 22 is provided to retain reagent containers 9d in which a dispersion (R4 reagent) is stored and reagent containers 9e in which a light-emitting substrate (R5 reagent) emitting light in a reaction process with the labeled antibody are stored two by two respectively (see FIG. 4), as shown in FIG. 1, and so formed that these reagent containers 9d and 9e are settable and exchangeable by the user. The second reagent set unit 22 lines up the reagent containers 9d and the reagent containers 9e in the direction X respectively and retains the same, and is formed to be capable of sucking the R4 reagent and the R5 reagent with the third reagent dispensing arm 23 respectively through two openings 221 and 222 provided on the upper surface of the second reagent set unit 22 in correspondence to the reagent containers 9d and the reagent containers 9e. FIG. 1 shows a state where the reagent containers 9d and 9e are drawn out of the second reagent set unit 22 for the purpose of illustration.

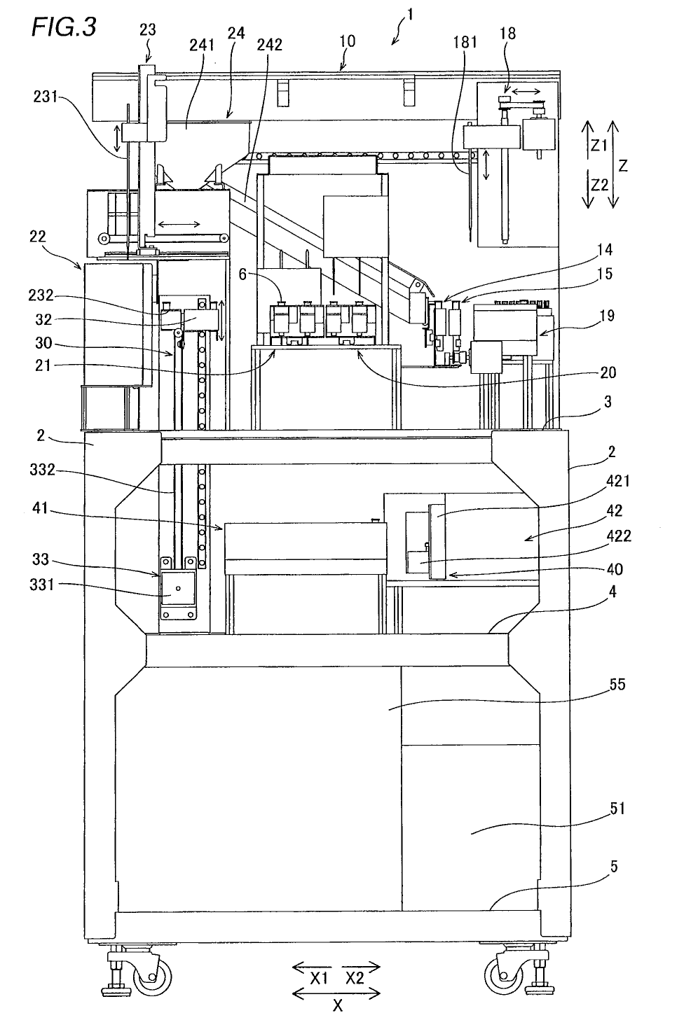

The third reagent dispensing arm 23 has a function for dispensing the reagents (the R4 reagent and the R5 reagent) in the reagent containers 9d and the reagent containers 9e on the second reagent set unit 22 into the cuvettes 6, as shown in FIGS. 3 and 4. This third reagent dispensing arm 23 is formed to be movable above the second reagent set unit 22 (openings 221 and 222), the cuvette retention portion 232 (R4 reagent dispensing position) and retention holes 31 (R reagent dispensation position), described later, of the container transfer portion 30 in the direction X, and has a pipette 231 (see FIG. 3) movable in the vertical direction (direction Z). The third reagent dispensing arm 23 is formed to suck the R4 reagent from the reagent containers 9d with the pipette 231 through the opening 221 (see FIG. 2) of the second reagent set unit 22 and to dispense the R4 reagent into the cuvettes 6 set on the cuvette retention portion 232. Further, the third reagent dispensing arm 23 is formed to suck the R5 reagent from the reagent containers 9e with the pipette 231 through the opening 222 (see FIG. 2) and to dispense the R5 reagent into the cuvettes 6 set in the retention holes 31 of the container transfer portion 31.

As shown in FIG. 4, the cuvette supply portion 24 has a cuvette introduction portion 241 into which the cuvettes 6 are introduced by the user, and has a function of successively supplying the cuvettes 6 up to an end portion position of a transport lane 242 transporting the cuvettes 6 to prescribed positions.

The cuvettes 6 supplied by the cuvette supply portion 24 are formed to be transferred to the first cuvette transport portion 14, the second cuvette transport portion 15 and the antigen-antibody reaction table 19 by a catcher 25a (see FIG. 4) movable in the direction X, the direction Y and the direction Z. The immunoanalyzer 1 is so formed that transfer of the cuvettes 6 to the primary BF separation portion 20, the secondary BF separation portion 21, the cuvette retention portion 232 and the container transfer portion 30 is performed by a catcher 25b (see FIG. 4) movable in the direction X and the direction Z.

According to this embodiment, the container transfer portion 30 includes a set portion 32 having the retention holes 31 and a raising/lowering mechanism 33 for raising/lowering the set portion 32 in the vertical direction (direction Z), as shown in FIGS. 2 and 3. The set portion 32 has two retention holes 31, and is formed to be capable of inserting the cuvettes 6 into the retention holes 31 and retaining the same. According to this embodiment, the retention holes 31 of the set portion 32 are arranged to line up with the opening 221 of the second reagent set unit 22 and the third reagent dispensing arm 23 in the direction X, and the immunoanalyzer 1 is formed to be capable of performing dispensation of the R5 reagent into the cuvettes 6 in the retention holes 31 with the third reagent dispensing arm 23 in a state setting the cuvettes 6 in the retention holes 31. The raising/lowering mechanism 33 is formed to transport (raise/lower) the set portion 32 from the upper layer U to the middle layer M with a motor 331 set on the second base 4 and a driving belt 332 provided from an upper end portion of the container transfer portion 30 on the first base 3 to the motor 331 of the second base 4. Thus, it is possible to transfer the cuvettes 6 into which the samples and all reagents from the R1 reagent up to the R5 reagent have been dispensed from the first sample processing portion 10 on the first base 3 to the second sample processing portion 40 on the second base 4 downward (Z2 direction). A passing hole 3a for passing the set portion 32 therethrough is provided on the first base 3, as shown in FIG. 2.

The second sample processing portion 40 on the second base 4 is formed to carry out other processes other than the processes having been carried out by the first sample processing portion 10 among the plurality of processes carried out by the immunoanalyzer 1 on the samples in the cuvettes 6, and includes an enzyme reaction portion 41 and the detection portion 42, as shown in FIG. 5. A fluid portion 43 including an electromagnetic valve for controlling supply and disposal paths for various fluids such as the washing solution, a pump for performing suction and discharge of the samples, the reagents etc. and the like is arranged on the second base 4, in addition to the second sample processing portion 40. FIGS. 1 to 3 omit illustration of this fluid portion 43.

The enzyme reaction portion 41 is provided for performing enzyme reaction (reaction 4) between the (enzyme-) labeled antibody (R3 reagent) in reaction specimens after antigen-antibody reaction (the reaction 1 to the reaction 3) and the light-emitting substrate (R5 reagent). A plurality of storage holes 411 for retaining the cuvettes 6 and performing incubation are provided on the enzyme reaction 41 in the form of a column in the direction X.

The detection portion 42 is an optical detection unit having a function of detecting light generated in a reaction process between the labeled antibody (R3 reagent) bound to the antigens in the samples and the light-emitting substrate (R5 reagent) with a photomultiplier tube (Photo Multiplier Tube) thereby measuring the quantities of the antigens contained in the samples. This detection portion 42 includes an openable/closable lid 421 and a set portion 422 capable of getting into/out of the detection portion 42 by moving in the direction Y. The detection portion 42 is so formed that the cuvettes 6 after the enzyme reaction (reaction 4) process with the enzyme reaction portion 41 are set on the set portion 42 and the cuvettes 6 are incorporated into the detection portion 42 whereby the measurement of the quantities of the antigens is performed in the detection portion 42. The set portion 422 is provided with a magnet 423 (see FIG. 10) for collecting the magnetic particles in the cuvettes 6.

Transfer of the cuvettes 6 in the second sample processing portion 40 on the second base 4 is performed by a catcher 44. The catcher 44 is formed to be capable of transferring the cuvettes 6 between the retention holes 31 of the container transfer portion 30 arranged to line up in the direction X, the storage holes 411 of the enzyme reaction portion 41 and the set portion 422 of the detection portion 42.

As shown in FIG. 6, the third base 5 of the lowermost portion is provided with various set portions including washing solution set portions 51 and 52 on which washing solution containers storing various washing solutions are settable respectively, a power source set portion 53 on which a power supply unit performing power supply to the respective portions is settable, a computer set portion 54 on which a measurement control portion 60a described later is settable, an air pressure source set portion 55 on which an air pressure source supplying positive pressure or negative pressure when performing suction and discharge of the samples, the reagents and the washing solutions etc. is settable, and a further apparatus set portions 56. A disposal box set portion 57 on which a disposal box for discarding the pipette chips 8 is settable is provided above the washing solution set portion 51 and the power source set portion 53. FIGS. 1 to 3 partially or entirely omit a power source, the air pressure source etc. set on these set portions.

As shown in FIG. 12, the immunoanalyzer 1 is provided with a body cover 27 covering the inner portion of the upper layer U, an outer cover 28 covering the inner portion of the middle layer M and another outer cover 29 covering the inner portion of the lower layer L. The body cover 27 and the outer covers 28 and 29 are made of materials having light blocking effects respectively, and hence the inner portions of the upper layer U, the middle layer M and the lower layer L enter blocked states in a state where the body cover 27 covers the inner portion of the upper layer U. Therefore, not only external light hardly reaches the inner portion of the middle layer M from above the first base 3 due to the first base 3 and respective units on the first base 3, but also the inner portion of the middle layer M is blocked (shielded) by the body cover 27 and the outer covers 28 and 29, whereby the inner portion of the middle layer M can be brought into a dark state. Therefore, it becomes possible to more precisely perform detection of light by the detection portion 42.

The body cover 27 is formed to be rotatable on a rotation axis 27a (see the one-dot chain line), whereby the inner portion of the upper layer U is openable/closable. In order to improve workability for the user, the immunoanalyzer 1 is so formed that the user can access respective units of the first sample processing portion 10 when the body cover 27 is opened. More specifically, the immunoanalyzer 1 is so formed that there exists a space where the user can set the rack 7a on the rack set portion 11 from above the sample rack set portion 11, there exists a space where the user can set the chip rack 121 on the chip rack set portion 12 from above the chip rack set portion 12, there exist spaces where the user can set the reagent containers on the respective ones of the first reagent set unit 16 and the second reagent set unit 22 from above the respective ones of the first reagent set unit 16 and the second reagent set unit 22 and there exists a space where the user can introduce the cuvettes 6 (see FIG. 2) into the cuvette introduction portion 241 from above the cuvette introduction portion 241 when the body cover 27 is opened, as shown in FIG. 1. As shown in FIG. 12, the outer covers 28 and 29 are provided to be easily detachable so that maintenance of units arranged on the middle layer M, setting of the washing solution containers on the lower layer L etc. can be easily performed.

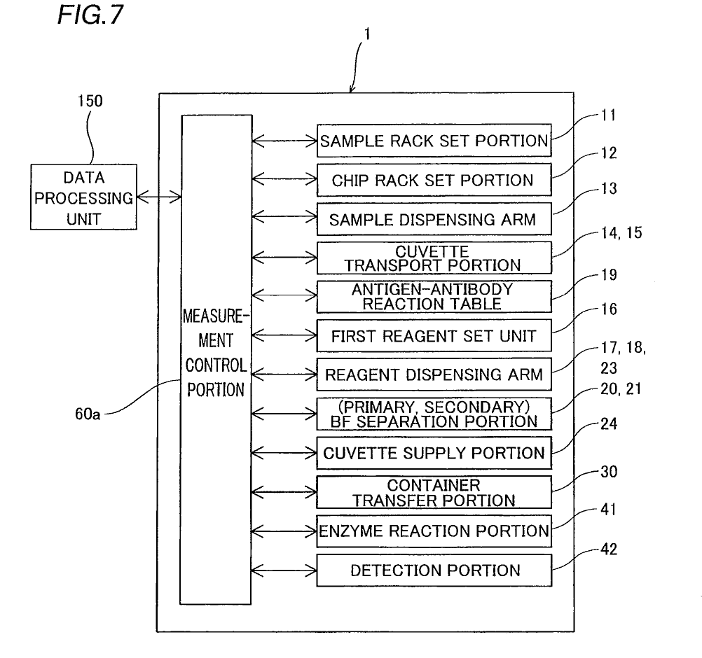

The respective mechanisms (various dispensing arms, the first BF separation portion 20, the second BF separation portion 21 and the raising/lowering mechanism 33 etc.) in the first sample processing portion 10, the container transfer portion 30 and the second sample processing portion 40 are controlled by the measurement control portion 60a, as shown in FIG. 7.

As shown in FIG. 8, the measurement control portion 60a is mainly constituted of a CPU 60b, a ROM 60c, a RAM 60d, an input/output interface 60e and a communication interface 60f. The CPU 60b, the ROM 60c, the RAM 60d, the input/output interface 60e and the communication interface 60f are connected with each other by a bus 60g.

The CPU 60b is capable of running computer programs stored in the ROM 60c and a computer program read on the RAM 60d. The ROM 60c stores the computer programs to be run by the CPU 60b and data employed for running the computer programs etc. The RAM 60d is employed for reading out the computer programs stored in the ROM 60c, and utilized as a working area of the CPU 60b when running these computer programs.

The input/output interface 60e is constituted of a parallel interface and an analog interface etc., for example. A bar code reader 61 is connected to the input/output interface 60e. Bar codes recording information for specifying the samples in the test tubes 7 and the rack 7a are assigned to the test tubes 7 storing the samples and the rack 7a on which the plurality of test tubes 7 are placed, and the bar coder reader 61 has a function of reading the bar codes assigned to these test tubes 7 and the rack 7a.

The communication interface 60f is an Ethernet (registered trademark) interface, for example. The communication interface 60f is so formed that data can be transferred/received between the measurement control portion 60a and the data processing unit 150 by using a prescribed communication protocol.

The data processing unit 150 consists of a personal computer (PC) or the like, and includes a control portion 150a (PC body) consisting of a CPU, a ROM, a RAM and the like, a display portion 150b and a keyboard 150c. The display portion 150b is provided for displaying analytical results or the like obtained by analyzing data of digital signals transmitted from the measurement control portion 60a.

Various computer programs such as an operating system and an application program for immunoassay etc. and data employed for running the computer programs are installed in the control portion 150a. The control portion 150a runs this application program for immunoassay, thereby measuring the quantities of the antigens or the antibodies in the measurement specimens on the basis of the quantities of light emission (data of digital signals) of the measurement specimens transmitted from the detection portion 42.

Processes of the immunoanalyzer 1 according to the embodiment of the present invention are now described with reference to FIGS. 1 to 5 and FIGS. 9 to 11. As described above, operation control of the respective mechanisms (respective dispensing arms, the primary BF separation portion 20, the secondary BF separation portion 21 and the raising/lowering mechanism 33 etc.) of the first sample processing portion 10, the container transfer portion 30 and the second sample processing portion 40 is performed by the measurement control portion 60a. Among a plurality of processes (“incubation process (reaction 1)”, “R2 reagent dispensing process”, “incubation process (reaction 2)”, “first washing process in the primary BF separation portion 20”, “stirring process in the primary BF separation portion 20”, “second washing process in the primary BF separation portion 20”, “R3 reagent dispensing process”, “incubation process (reaction 3)”, “first washing process, stirring process and second washing process in the secondary BF separation portion 21”, “R4 reagent dispensing process”, “R5 reagent dispensing process”, “incubation process (reaction 4)” and “measuring process” described below) carried out by the immunoanalyzer 1 on the samples in the cuvettes 6, the processes from the “incubation process (reaction 1)” up to the “R5 reagent dispensing process” are carried out in the first sample processing portion 10, and the “incubation process (reaction 4)” and the “measuring process” are carried out in the second sample processing portion 40.

### (Cuvette Supply Process)

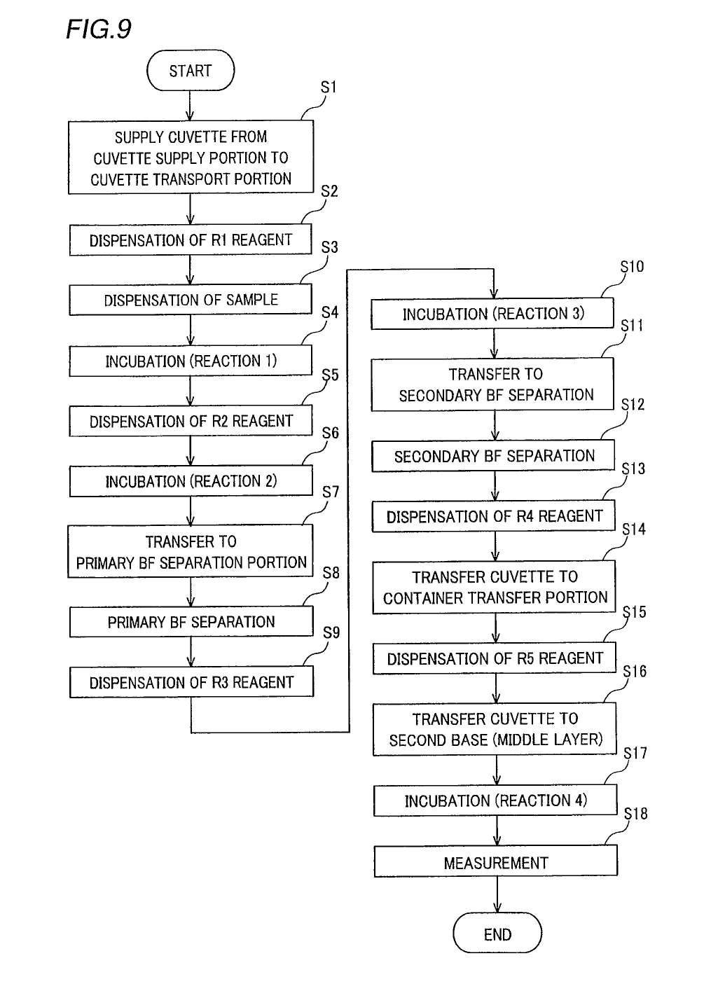

At a step S1 in FIG. 9, each cuvette 6 is supplied to the end portion position of the transportation lane 242 of the cuvette supply portion 24 and transported to the first cuvette transport portion 14 by the catcher 25a, as shown in FIG. 4. The cuvette 6 is set in the cuvette receiving hole 141 of the first cuvette transport portion 14.

### (R1 Reagent Dispensing Process)

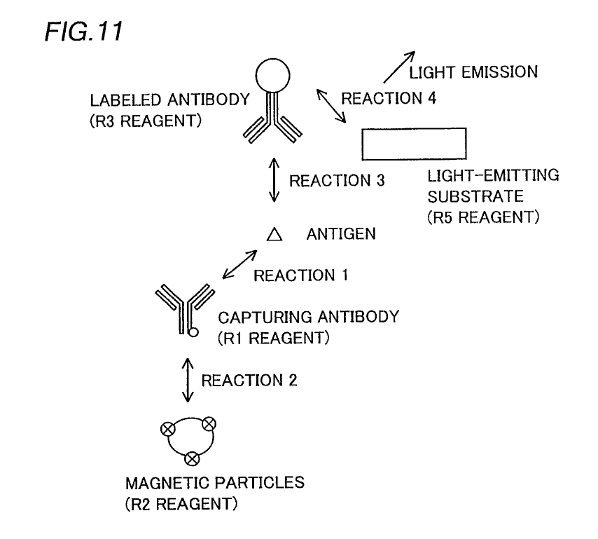

At a step S2, a prescribed quantity or R1 reagent is dispensed into the cuvette 6 set in the cuvette receiving hole 141 of the first cuvette transport portion 14. In other words, the cuvette 6 retained in the cuvette receiving hole 141 of the first cuvette transport portion 14 is moved to the R1 reagent dispensation position P1, while the R1/R3 set portion 161 of the first reagent set unit 16 moves in a Y1 direction and the reagent container 9a storing the R1 reagent is arranged on the sucking position P21. Further, the first reagent dispensing arm 17 moves up to a portion above the first reagent set unit 16, and the R1 reagent stored in the corresponding reagent container 9a is sucked by the pipette 171 through the corresponding hole portion 163a (see FIG. 1). Then, the first reagent dispensing arm 17 moves in the arrow X1 direction up to the R1 reagent dispensation position P1, and the R1 reagent is dispensed (discharged) from the pipette 171 into the cuvette 6 set in the cuvette receiving hole 141. As shown in FIGS. 10 and 11, the capturing antibody bound to the antigen contained in each sample is contained in the R1 reagent.

### (Sample Dispensing Process)

Then, the cuvette 6 set in the cuvette receiving hole 141 of the first cuvette transport portion 14 is moved to the sample dispensation position P2, while a prescribed quantity of the sample is dispensed into this cuvette 6 at a step S3, as shown in FIG. 6. At this time, the corresponding pipette chip 8 (see FIG. 1) retained on the chip rack 121 is mounted on the pipette portion 131 of the sample dispensing arm 13, while the sample dispensing arm 13 moves in the arrow X2 direction, and the sample such as blood is sucked from the test tube 7 retained in the rack 7a on the lateral feed portion 113 of the sample rack set portion 11 by the pipette portion 131. Thereafter the sample dispensing arm 13 moves to the sample dispensation position P2, and the sample is dispensed (discharged) into the cuvette 6 (the cuvette 6 into which the R1 reagent has been dispensed) in the cuvette receiving hole 141 from the pipette portion 131.

### (Incubation Process (Reaction 1 Shown in FIGS. 10 and 11)

At a step S4, the first cuvette transport portion 14 is moved in the arrow Y1 direction up to a side portion of the antigen-antibody reaction table 19, and the cuvette 6 in the cuvette receiving hole 141 is transferred to the corresponding storage hole 191 of the first reaction portion 192 by the catcher 25a. When extracting the cuvette 6 into which the R1 reagent and the sample have been dispensed from the cuvette receiving hole 141, the catcher 25 stirs the specimen in the cuvette 6, and thereafter sets the same in the storage hole 191 of the first reaction portion 192. The R1 reagent and the sample as stirred are incubated for a prescribed time in the cuvette 6 retained in the receiving hole 191 of the first reaction portion 192 of the antigen-antibody reaction table 19. Thus, the capturing antibody (R1 reagent) and the antigen in the sample are bound to each other (reaction 1).

### (R2 Reagent Dispensing Process)

At a step S5, the cuvette 6 after the reaction (reaction 1) is set in the cuvette receiving hole 151 of the second cuvette transport portion 15 by the catcher 25a, thereafter the cuvette 6 retained in the cuvette receiving hole 151 of the second cuvette transport portion 15 is moved up to the R2 reagent dispensation position P11, and a prescribed quantity of the R2 reagent is dispensed into this cuvette 6 by the second reagent dispensing arm 18, as shown in FIG. 4. In other words, the second reagent dispensing arm 18 moves up to the portion above the first reagent set unit 16 and the R2 reagent stored in the reagent container 9b is sucked by the pipette 181 through the hole portion 163b, while the second reagent dispensing arm 18 moves up to the R2 reagent dispensation position P11 and the R2 reagent is dispensed (discharged) into the cuvette 6 set in the cuvette receiving hole 151 from the pipette 181. Magnetic particles bound to the capturing antibody to which the antibody in the sample is bound are contained in the R2 reagent, as shown in FIGS. 10 and 11.

### (Incubation Process (Reaction 2 Shown in FIGS. 10 and 11)

At a step S6, the cuvette 6 set in the cuvette receiving hole 151 of the second cuvette transport portion 15 is extracted by the catcher 25a, stirred, and thereafter set in the storage hole 191 of the first reaction portion 192 of the antigen-antibody reaction table 19 again, as shown in FIG. 4. The R1 reagent, the sample and the R2 reagent as stirred are incubated for a prescribed time in the cuvette 6 retained in the storage hole 191 of the first reaction portion 192. Thus, the magnetic particles (R2 reagent) in the cuvette 6 and the capturing antibody (R1 reagent) to which the antigen in the sample is bound are bound to each other (reaction 2).

(Transfer Process from Antigen-Antibody Reaction Table 19 to Primary BF Separation Portion 20)

Thereafter the cuvette 6 storing the R1 reagent, the sample and the R2 reagent as incubated is transferred to the corresponding set hole 201 of the primary BF separation portion 20 at a step S7. First, the cuvette 6 storing the specimen after the reaction (reaction 2) is transferred from the storage hole 191 of the first reaction portion 192 to the cuvette receiving hole 142 of the first cuvette transport portion 14 by the catcher 25a, and transported to the first BF delivery position P3 by the first cuvette transport portion 14. The cuvette 6 in the cuvette receiving hole 142 is extracted by the catcher 25b on the first BF delivery position P3, moved in the arrow X2 direction and set in the set hole 201 of the primary BF separation portion 20.

Then, a primary BF separation process of separating an unreacted R1 reagent (unnecessary component) and the magnetic particles from the specimen (specimen after the reaction 1 and the reaction 2 have been performed) in the cuvette 6 set in the set hole 201 is performed by the primary BF separation portion 20 at a step S8. This BF separation process consists of a first washing process described below as well as four times of a stirring process and four times of a second washing process.

### (First Washing Process in First Primary BF Separation Portion 20)

First, the magnetic particles in the cuvette 6 retained on the set portion 201 are collected by the magnet 202 arranged on a side portion of the cuvette 6, as shown in FIG. 10. Then, the magnetic particles and the unnecessary component (liquid) excluding the antigen bound to the magnetic particles through the capturing antibody are removed by sucking the specimen in the cuvette 6 with a nozzle (not shown) of a washing mechanism (not shown). Thereafter the stirring process and the second washing process described below are carried out, in order to sufficiently remove the unnecessary component.

### (Stirring Process in Primary BF Separation Portion 20)

After a washing solution is supplied into the cuvette 6, on which the first washing process has been carried out, by the washing mechanism (not shown), the cuvette 6 is grasped by the stirring mechanism (not shown) and whirling vibration is applied thereto whereby stirring is performed. Thus, the washing solution, the unnecessary component and the magnetic particles in the cuvette 6 are stirred, and it becomes possible to disperse the unnecessary component (unnecessary component not completely removable in the first washing process) having remained on the inner wall of the cuvette 6 along with the magnetic particles. The nozzle (not shown) of the washing mechanism (not shown) is washed for suction for the second time during this stirring process.

### (Second Washing Process in Primary BF Separation Portion 20)

Then, after the magnetic particles in the cuvette 6 stirred by the stirring mechanism (not shown) of the primary BF separation portion 20 are collected to the side of the magnet 202 arranged on the side portion of the cuvette 6, the washing solution and the unnecessary component are discharged by the already washed nozzle of the washing mechanism (not shown). It becomes possible to remove the unnecessary component having been rolled in the magnetic particles to remain, by stirring and thereafter sucking the washing solution in the cuvette 6 in this manner. Thereafter the aforementioned stirring process and the second washing process are repeated by a prescribed number of times (three times), whereby the remaining unnecessary component is removed. Thus, removal of the unnecessary component by the first washing process as well as the four times of the stirring process and the four times of the second washing process is performed in the primary BF separation process.

### (R3 Reagent Dispensing Process)

Thereafter a prescribed quantity of the R3 reagent is dispensed into the cuvette 6, in which the separation of the unnecessary component and the magnetic particles has been performed by the primary BF separation portion 20, at a step S9. First, the cuvette 6 is extracted from the set hole 201 of the primary BF separation portion 20 by the catcher 25b, and set in the cuvette receiving hole 153 of the second cuvette transport portion 15 on the second BF delivery position P13, as shown in FIG. 4. Then, the cuvette 6 retained in the cuvette receiving hole 153 of the second cuvette transport portion 15 is moved to the R3 reagent dispensation position P12, while the R1/R3 set portion 161 moves and the reagent container 9c storing the R3 reagent is arranged on the sucking position P21. Further, the first reagent dispensing arm 17 moves up to the portion above the first reagent set unit 16, and the R3 reagent stored in the reagent container 9c is sucked by the pipette 171 through the hole portion 163a. Then, the first reagent dispensing arm 17 moves in the arrow X1 direction up to the R3 reagent dispensation position P12, and the R3 reagent is dispensed (discharged) into the cuvette 6 set in the cuvette receiving hole 153 from the pipette 171. As shown in FIGS. 10 and 11, the (enzyme-) labeled antibody bound to the antigen in the sample is contained in the R3 reagent.

### (Incubation Process (Reaction 3 Shown in FIGS. 10 and 11))

At a step S10, the second cuvette transport portion 15 is moved in the arrow Y1 direction up to the side portion of the antigen-antibody reaction table 19, and the cuvette 6 in the cuvette receiving hole 153 is transferred to the storage hole 191 of the second reaction portion 193 by the catcher 25a, as shown in FIG. 4. When extracting the cuvette 6 into which the sample, the R1 reagent, the R2 reagent and the R3 reagent have been dispersed from the cuvette receiving hole 153, the catcher 25a stirs the specimen in the cuvette 6, and thereafter sets the same in the storage hole 191 of the second reaction portion 193. The R3 reagent containing the capturing antibody (R1 reagent), the antigen (sample), the magnetic particles (R2 reagent) and the labeled antibody as stirred is incubated for a prescribed time in the cuvette 6 retained in the storage hole 191 of the second reaction portion 193 of the antigen-antibody reaction table 19. Thus, the antigen bound to the magnetic particles (R2 reagent) through the capturing antibody (R1 reagent) and the labeled antibody (R3 reagent) are bound to each other (reaction 3).

(Transfer Process from Antigen-Antibody Reaction Table 19 to Secondary BF Separation Portion 21)

At a step S11, the cuvette 6 storing the R3 reagent containing the capturing antibody (R1 reagent), the antigen (sample), the magnetic particles (R2 reagent) and the labeled antibody as incubated is transferred to the set hole 211 of the secondary BF separation portion 21. First, the cuvette 6 storing the specimen after the reaction (reaction 3) is transferred from the storage hole 191 of the second reaction portion 193 to the cuvette receiving hole 152 of the secondary cuvette transport portion 15 by the catcher 25a, and transported up to the second BF delivery position P13 by the second cuvette transport portion 15, as shown in FIG. 4. Then, the cuvette 6 in the cuvette receiving hole 152 is extracted by the catcher 25b on the second BF delivery position P13, moved in the arrow X2 direction and set in the set hole 211 of the secondary BF separation portion 21.

### (First Washing Process, Stirring Process and Second Washing Process in Secondary BF Separation Portion 21)

Then, a secondary BF separation process consisting of a first washing process as well as four times of a stirring process and four times of a second washing process is carried out in the secondary BF separation portion 21 at a step S12 as shown in FIG. 10, similarly to the primary BF separation process (see the step S8) in the aforementioned primary BF separation portion 20. Thus, it becomes possible to perform sufficient removal of the R3 reagent (unnecessary component) containing the labeled antibody not bound to the antigen in the sample. The contents of the secondary BF separation process are similar to those of the aforementioned primary BF separation process.

### (R4 Reagent Dispensing Process)

Thereafter the R4 reagent (dispersion) is dispensed into the cuvette 6 storing the specimen containing the antigen to which the labeled antibody from which the unnecessary component has been removed is bound at a step S13. First, the cuvette 6 after completion of the secondary BF separation process is extracted from the set hole 211 of the second BF separation portion 21 by the catcher 25b, moved in the arrow X2 direction and set in the cuvette retention portion 232, as shown in FIG. 4. Further, the third reagent dispensing arm 23 moves up to a portion above the second reagent set unit 22 and the R4 reagent stored in the reagent container 9d is sucked by the pipette 231 through the opening 221 (see FIG. 2), while the third reagent dispensing arm 23 moves to a portion (R4 reagent dispensing position) above the cuvette retention portion 232, and the R4 reagent is dispensed (discharged) into the cuvette 6 set in the cuvette retention portion 232 from the pipette 231.

(Transfer Process from Cuvette Retention Portion 232 to Container Transfer Portion 30)

After the dispensation of the R4 reagent, the cuvette 6 into which the R4 reagent has been dispensed is set in the corresponding retention hole 31 provided on the set portion 32 of the container transfer portion 30. In other words, the cuvette 6 into which the R4 reagent has been dispensed is extracted from the cuvette retention portion 232 by the catcher 25b, moved in the arrow X1 direction and transferred to the adjacent retention hole 31 of the container transfer portion 30.

### (R5 Reagent Dispensing Process)

At a step S15, the R5 reagent containing the light-emitting substrate is dispensed into the cuvette 6 retained on the set portion 32 (retention hole 31) of the container transfer portion 30. In other words, the third reagent dispensing arm 23 moves up to the portion above the second reagent set unit 22 and the R5 reagent stored in the reagent container 9e is sucked by the pipette 231 through the opening 222 (see FIG. 2), while the third reagent dispensing arm 23 moves up to a portion (R5 reagent dispensing position) above the retention hole 31 of the container transfer portion 30 and the R5 reagent is dispensed (discharged) into the cuvette 6 set on the container transfer portion 30 from the pipette 231. As shown in FIGS. 10 and 11, the light-emitting substrate emitting light by reacting with the labeled antibody in the R3 reagent is contained in the R5 reagent.

(Downward Transfer Process from Upper Layer U to Middle Layer M)

When the R5 reagent is dispensed into the cuvette 6 on the set portion 32 of the container transfer portion 30, the cuvette 6 retained on the set portion 32 of the container transfer portion 30 is transferred from the upper layer U to the middle layer M at a step S16. When the R5 reagent is dispensed into the cuvette 6 on the set portion 32, the raising/lowering mechanism 33 is so driven that the set portion 32 is lowered downward (arrow Z2 direction) while retaining the cuvette 6 and transferred up to a prescribed position in the middle layer M according to this embodiment, as shown in FIG. 3.

(Incubation Process (Reaction 4 shown in FIGS. 10 and 11))

Then, the cuvette 6 on the container transfer portion 30 is extracted from the set portion 32 (retention hole 31) of the container transfer portion 30 by the catcher 44 at a step S17, and the specimen in the cuvette 6 is stirred and thereafter set in the storage hole 411 of the enzyme reaction portion 41, as shown in FIG. 5. The capturing antibody (R1 reagent), the antigen (sample), the magnetic particles (R2 reagent), the labeled antibody and the R5 reagent, containing the light-emitting substrate, as stirred are incubated for a prescribed time in the cuvette 6 set in the storage hole 411 of the enzyme reaction portion 41. Thus, reaction (reaction 4) between the labeled antibody (R3 reagent) and the light-emitting substrate (R5 reagent) progresses.

### (Measuring Process)

Thereafter the cuvette 6 storing the capturing antibody (R1 reagent), the antigen (sample), the magnetic particles (R2 reagent), the labeled antibody and the R5 reagent, containing the light-emitting substrate, as incubated is extracted from the storage hole 411 of the enzyme reaction portion 41 by the catcher 44, and transferred to the set portion 422 of the detection portion 42 at a step S18. When the cuvette 6 is set on the set portion 422, the set portion 422 moves in the arrow Y2 direction and the cuvette 6 is incorporated into the detection portion 42, while the openable/closable lid 421 is closed. Then, the sample is analyzed by acquiring the quantity of light emission caused in a reaction process between the labeled antibody in the R3 reagent and the light-emitting substrate in the R5 reagent by the photomultiplier tube (not shown) in the detection portion 42, as shown in FIG. 11. At this time, the magnetic particles in the cuvette 6 set on the set portion 422 are attracted to the side of the magnet 423, as shown in FIG. 10. Thus, the magnetic particles are inhibited from hindering the measurement of the quantity of light emission when measuring the quantity of light emission caused in the reaction process between the labeled antibody in the R3 reagent and the light-emitting substrate in the R5 reagent. An analytical operation of the immunoanalyzer 1 according to the embodiment is performed in the aforementioned manner.

According to this embodiment, as hereinabove described, the first sample processing portion 10 is set on the first base 3 and the second sample processing portion 40 is set on the second base 4 arranged under the first base 3, and the container transfer portion 30 that transfers the cuvettes 6 from the upper layer U to the middle layer M is provided, whereby a plurality of units for carrying out the plurality of processes respectively can be arranged dividedly to the first sample processing portion 10 of the first base 3 and the second sample processing portion 40 of the second base 4 arranged in the vertical direction (direction Z), and transfer of the cuvettes 6 between the upper layer U and the middle layer M can be performed by the container transfer portion 30. Thus, the immunoanalyzer 1 can be inhibited from enlarging in the horizontal direction (direction XY) also in a case where a large number of units must be set in the immunoanalyzer 1, and the process can be smoothly performed also in the case of vertically dividedly arranging the plurality of units. Consequently, a set area of the immunoanalyzer 1 can be reduced while smoothly performing the process.

According to this embodiment, as hereinabove described, the dimension of the immunoanalyzer 1 in the horizontal direction (Direction XY) can be reduced by vertically arranging the first base 3 and the second base 4 to completely overlap with each other in plan view, whereby the immunoanalyzer 1 can be easily miniaturized.

According to this embodiment, as hereinabove described, the first sample processing portion 10 is arranged on the upper layer U which is the uppermost layer, while the first reagent set unit 16 and the second reagent set unit 22, the first reagent dispensing arm 17, the second reagent dispensing arm 18 and the third reagent dispensing arm 23 are provided on the first sample processing portion 10. Thus, the user's access to the first sample processing portion 10 is simplified, whereby the user can easily set the reagent containers 9a to 9e storing the R1 reagent to the R5 reagent on the first reagent set unit 16 and the second reagent set unit 22 respectively.

According to this embodiment, as hereinabove described, the first sample processing portion 10 is arranged on the upper layer U which is the uppermost layer, while the sample rack set portion 11 and the sample dispensing arm 13 are provided on the first sample processing portion 10. Thus, the user's access to the first sample processing portion 10 is simplified, whereby the user can easily set the test tubes 7 on the sample rack set portion 11.

According to this embodiment, as hereinabove described, the first sample processing portion 10 is arranged on the upper layer U which is the uppermost layer, while the cuvette supply portion 24, the sample dispensing arm 13, the first reagent dispensing arm 17, the second reagent dispensing arm 18 and the third reagent dispensing arm 23 are provided on the first sample processing portion 10. Thus, the user's access to the first sample processing portion 10 is simplified, whereby the user can easily introduce the cuvettes 6 into the cuvette supply portion 24.

According to this embodiment, as hereinabove described, the sample dispensing arm 13, the first reagent dispensing arm 17, the second reagent dispensing arm 18 and the third reagent dispensing arm 23 and the antigen-antibody reaction table 19 for carrying out the processes (the reaction 1 to the reaction 3) of reacting the sample in the cuvette 6 with the R1 reagent, the R2 reagent and the R3 reagent are provided on the first sample processing portion 10 of the first base 3 while the enzyme reaction portion 41 for carrying out the process (reaction 4) of reacting the specimen in the cuvette 6 and the R5 reagent with each other and the detection portion 42 are provided on the second sample processing portion 40 of the second base 4, and the immunoanalyzer 1 has been formed to transfer the cuvette 6 into which the R1 reagent to the R3 reagent, the R4 reagent and the R5 reagent have been dispensed by the first reagent dispensing arm 17, the second reagent dispensing arm 18 and the third reagent dispensing arm 23 of the first sample processing portion 10 to the middle layer M with the container transfer portion 30. The immunoanalyzer 1 is so formed in this manner that the first sample processing portion 10 carries out the respective dispensing processes of dispensing the R1 reagent to the R3 reagent into the cuvette 6, the respective reactions processes (the reaction 1 to the reaction 3) between the sample and the R1 reagent to the R3 reagent and the respective dispensations processes of dispensing the R4 reagent and the R5 reagent into the cuvette 6, and the cuvette 6 in which no further reagent may be added to the specimen in subsequent processes can be transferred to the middle layer M by the container transfer portion 30. Thus, it becomes unnecessary to set reagent dispensing arms on the second base 4 (second sample processing portion 40). Further, the process (reaction 4) of reacting the specimen and the R5 reagent with each other can be carried out in the second sample processing portion 40 after performing the dispensation of the R1 reagent to the R5 reagent on the first base 3 (first sample processing portion 10), whereby the number of units set on the first base 3 (first sample processing portion 10) can be reduced due to the provision of the enzyme reaction portion 41 and the detection portion 42 on the second base 4.

According to this embodiment, as hereinabove described, the immunoanalyzer 1 is so formed that the third reagent dispensing arm 23 dispenses the R5 reagent into the cuvette 6 retained on the container transfer portion 30, whereby the cuvette 6 can be immediately transferred from the upper layer U to the middle layer M after completion of the dispensation of the R5 reagent with the third reagent dispensing arm 23.

According to this embodiment, as hereinabove described, the detection portion 42 consisting of the optical detection unit is provided on the second sample processing portion 40 of the second base 4 provided under (arrow Z2 direction) the first base 3 so that the detection portion 42 (optical detection unit) can be arranged on the lower second base 4 to which external light hardly reaches due to the first base 3 and the respective units on the first base 3, whereby the detection portion 42 (optical detection unit) can be arranged on a darker position. Thus, detection of light emitted from measurement specimens with the detection portion 42 (optical detection unit) can be more precisely performed.

According to this embodiment, as hereinabove described, the third base 5 is provided under the first base 3 and the second base 4 and the washing solution set portions 51 and 52 for setting liquid containers storing liquids such as washing solutions used by the first sample processing portion 10 and the second sample processing portion 40 are provided on the third base 5 so that the liquid containers storing the washing solutions can be set on the third base 5 arranged under the first base 3 and the second base 4, whereby the user may not raise the heavy liquid containers up to the positions of upper layers (the upper layer U and the middle layer M). Also in a case where the liquids spill out of the liquid containers in exchange of the liquid containers or the like, the liquids can be prevented from falling onto the respective units of the first base 3 (first sample processing portion 10) and the second base 4 (second sample processing portion 40).

The embodiment disclosed this time must be considered illustrative in all points and not restrictive. The range of the present invention is shown not by the above description of the embodiment but by the scope of claims for patent, and all modifications within the meaning and range equivalent to the scope of claims for patent are included.

For example, while the example of applying the sample analysis device according to the present invention to the immunoanalyzer 1 has been shown in the aforementioned embodiment, the present invention is not restricted to this. The present invention is applicable to any device so far as the same is a device carrying out a plurality of processes on a sample in a container, and applicable to a blood coagulation analyzer, a urine sample measuring device, a gene amplification detector or the like, in addition to the immunoanalyzer.

While the example of transferring the cuvettes 6 from the first sample processing portion 10 to the second sample processing portion 40 on the second base 4 by the container transfer portion 30 after completion of the processes by the first sample processing portion 10 on the first base 3 has been shown in the aforementioned embodiment, the present invention is not restricted to this. The third base may be arranged under the second base 4 to set the third sample processing portion on the third base, and the cuvettes 6 may be transferred to the third sample processing portion on the third base by the container transfer portion 30 after completion of the processes with the second sample processing portion 40. The cuvettes 6 may be transferred from the second sample processing portion 40 to the third sample processing portion with another container transfer portion different from the container transfer portion 30. Further, the cuvettes 6 may be transferred to the third sample processing portion with the container transfer portion 30 after completion of the processes by the first sample processing portion 10, and the cuvettes 6 may be transferred to the second sample processing portion 40 with the container transfer portion 30 after completion of the processes with the third sample processing portion.

The present invention may have such a structure that a processing unit that carries out further processes on the sample in the cuvette 6 other than the processes carried out by the immunoanalyzer 1 is further arranged on the first base 3 or the second base 4, or may have such a structure that a prescribed processing unit included in the immunoanalyzer 1 is omitted from the first base 3 or the second base 4.

While the example of arranging the enzyme reaction portion 41 and the detection portion 42 on the second base 4 has been shown in the aforementioned embodiment, the present invention is not restricted to this. According to the present invention, units other than the enzyme reaction portion and the detection portion may be arranged on the second base, and the third reagent dispensing arm 23 and the second reagent set unit 22 may be set on the second base 4, for example.

While the components in the measurement specimens are detected by incorporating the cuvettes 6 storing the measurement specimens into the detection portion 42 in the aforementioned embodiment, the present invention is not restricted to this. For example, detection of the components in the measurement specimens may be performed by transferring the measurement specimens stored in the cuvettes 6 into the detection portion with a pipette or a tube.

While the example of bringing the immunoanalyzer 1 into the three-layer structure consisting of the upper layer U, the middle layer M and the lower layer L has been shown in the aforementioned embodiment, the present invention is not restricted to this. According to the present invention, the immunoanalyzer 1 may be brought into a structure of at least four layers by further providing other layers, or may be brought into a two-layer structure consisting of an upper layer and a lower layer.

While the example of forming the first base 3, the second base 4 and the third base 5 in identical shapes and arranging the same in the vertical direction to completely overlap with each other in plan view has been shown in the aforementioned embodiment, the present invention is not restricted to this. For example, the respective bases may be shifted from each other and vertically arranged to partially overlap with each other. Further, any base may be formed to be larger than the remaining bases.

While the example of forming the immunoanalyzer 1 to transfer the cuvettes 6 to the middle layer M in the state where the cuvettes 6 are retained by the retention holes 31 of the container transfer portion 30 has been shown in the aforementioned embodiment, the present invention is not restricted to this. According to the present invention, a chuck member or the like may be provided on the container transfer portion, and the immunoanalyzer 1 may be formed to transfer the cuvettes to the middle layer M in a state of grasping the cuvettes with the chuck member.

While the example of forming the immunoanalyzer 1 to transfer the cuvettes 6 to the middle layer M with the container transfer portion 30 after various processes in the first sample processing portion 10 on the first base 3 are terminated has been shown in the aforementioned embodiment, the present invention is not restricted to this. According to the present invention, the immunoanalyzer 1 may be formed to transfer the cuvettes to the middle layer M with the container transfer portion once and to thereafter return the same to the upper layer U again for continuing the processes. Alternatively, the immunoanalyzer 1 may be formed to start the processes from the middle layer M and to transfer the cuvettes to the upper layer U.

While the first sample processing portion 10 on the first base 3 carries out the processes from the cuvette supply process up to the R5 reagent dispensing process and the second sample processing portion 40 on the second base 4 carries out the incubation process (enzyme reaction) and the measuring process in the aforementioned embodiment, the present invention is not restricted to this. According to the present invention, the second sample processing portion on the second base 4 may carry out the processes from the cuvette supply process up to the R5 reagent dispensing process, and the first sample processing portion on the first base 3 may carry out the incubation process (enzyme reaction) and the measuring process after transferring the cuvettes to the upper layer U with the container transfer portion.

While the upper layer U, the middle layer M and the lower layer L are formed by the first base 3 (excluding a raising/lowering region of the set portion 32), the second base 4 and the third base 5 entirely formed in plate shapes with neither recess portions nor through-holes in the aforementioned embodiment, the present invention is not restricted to this. According to the present invention, only placing regions for respective units in the bases forming the respective layers may be formed in plate shapes, and through-holes and recess portions may be formed on portions other than the placing regions.

While prescribed units are placed on the respective upper surfaces of the first base 3, the second base 4 and the third base 5 in the aforementioned embodiment, the present invention is not restricted to this. According to the present invention, the prescribed units may simply be set on the upper layer U, the middle layer M and the lower layer L. For example, the prescribed units may be mounted on the lower surfaces of the bases, or the prescribed units may be suspended from the lower surfaces of the bases.

While the example of forming the container transfer portion 30 to transfer the cuvettes 6 in the vertical direction (direction Z) has been shown in the aforementioned embodiment, the present invention is not restricted to this. For example, the container transfer portion may be formed to raise/lower (transfer) the cuvettes in an oblique vertical direction, or may be formed to transfer the cuvettes in still another direction other than the vertical direction and the oblique vertical direction.

While the example of forming the raising/lowering mechanism 33 by the container transfer portion 30 of the motor 331 and the driving belt 332 has been shown in the aforementioned embodiment, the present invention is not restricted to this. According to the present invention, the raising/lowering mechanism may be constituted of a ball screw and a ball nut or may be constituted of a rack and a pinion mechanism, or another mechanism other than this may be employed.

In order to keep the temperature of specimen liquids in the cuvettes 6 at a constant level, adiabatic process may be performed on the inner wall of the container transfer portion 30, or a warming portion may be provided on the container transfer portion 30.

While the example of providing various set regions including the washing solution set portions 51 and 52, the power source set portion 53, the computer set portion 54 and the air pressure source set portion 55 and another set portion 56 on the third base 5 has been shown in the aforementioned embodiment, the present invention is not restricted to this. A set region other than the aforementioned various set portions may be provided, or no set regions may be provided. Further, the respective set portions may be arranged on arbitrary positions.

While the washing solution set portions 51 and 52 for setting the washing solution containers storing the washing solutions are provided on the third base 5 as one of liquid containers storing liquids used for analyzing the samples in the aforementioned embodiment, the present invention is not restricted to this. Set regions for setting liquid containers storing liquid such as reagents and diluents mixed into the samples may be provided on the third base 5 as liquid containers storing liquids used for analyzing the samples.

While the cuvettes are employed as the containers for storing the samples and the reagents in the aforementioned embodiment, the present invention is not restricted to this. The same may simply be containers capable of storing liquids, and forward ends of pipette chips having been employed for dispensation of samples may be heat-sealed by heat sealing, so that reagents are dispensed into the pipette chips whose forward ends are bound and transferred from the upper layer U to the middle layer M, for example.

While the body cover 27 covering the inner portion of the upper layer U is made of a material having a light blocking effect in addition to the outer cover 28 covering the inner portion of the middle layer M and the outer cover 29 covering the inner portion of the lower layer L thereby bringing the inner portion of the upper layer U, the inner portion of the middle layer M and the inner portion of the lower layer L into blocked states in the aforementioned embodiment, the present invention is not restricted to this. The immunoanalyzer 1 may be so formed that external light is transmitted into the inner portion of the upper layer U by preparing the body cover 27 covering the upper layer U from a material having translucency or providing no body cover 27. Also in this case, external light can be inhibited from reaching the inner portion of the middle layer M due to the first base 3, the respective units on the first base 3 and the outer covers 28 and 29, whereby the inner portion of the middle layer M can be brought into a blocked state. In this case, therefore, the user can easily confirm operations of the respective units on the first base 3 by visual observation, and detection by the detection portion 42 set in the inner portion of the middle layer M can be precisely performed. The inner portion of the middle layer M can be kept in a darker state by preparing the first base 3 from a material having a light blocking effect.

What is claimed is:

1. A sample analysis device that analyzes a sample by carrying out a plurality of processes on the sample in a container and has a plurality of layers, comprising:
a first sample processing portion that is arranged in a first layer and that is configured to carry out one part of the plurality of processes on the sample in the container;
a second sample processing portion that is arranged in a second layer positioned above or under the first layer and that is configured to carry out at least another part of the plurality of processes on the sample in the container, the one part of the plurality of processes having been carried out on the sample in the container; and
a container transfer portion configured to transfer the container, which contains the sample on which the one part of the plurality of processes has been carried out, from the first layer to the second layer.

2. The sample analysis device according to claim 1, further comprising:
a first base, and
a second base arranged above or under the first base, wherein
the first sample processing portion is arranged on the first base, and
the second sample processing portion is arranged on the second base.

3. The sample analysis device according to claim 1, wherein
the first layer and the second layer are so arranged that substantially all areas overlap with each other in plan view.

4. The sample analysis device according to claim 1, wherein
the first layer is an uppermost layer, and
the first sample processing portion includes:
a reagent set unit on which a reagent employed for analyzing the sample is set by a user, and
a reagent dispensing unit configured to carry out a process of dispensing the reagent set on the reagent set unit into the container.

5. The sample analysis device according to claim 1, wherein
the first layer is an uppermost layer, and
the first sample processing portion includes:
a sample set unit on which a sample container storing the sample is set by a user, and
a sample dispensing unit configured to carry out a process of dispensing the sample in the sample container set on the sample set unit into the container.

6. The sample analysis device according to claim 1, wherein
the first layer is an uppermost layer, and
the first sample processing portion includes:
a container set unit on which the container is set by a user, and
a dispensing unit configured to carry out a process of dispensing the sample or a reagent into the container.

7. The sample analysis device according to claim 1, wherein
the first sample processing portion includes:
a sample dispensing unit configured to carry out a process of dispensing the sample into the container, and
a reagent dispensing unit configured to carry out a process of dispensing a reagent into the container, and
the second sample processing portion includes no dispensing unit configured to carry out a process of dispensing the sample or the reagent into the container.

8. The sample analysis device according to claim 1, wherein
the first sample processing portion includes:
a sample dispensing unit configured to carry out a process of dispensing the sample into the container,
a reagent dispensing unit configured to carry out a process of dispensing a reagent into the container, and
a first reaction unit configured to carry out a process of reacting the sample with one reagent in the container,
the second sample processing portion includes:
a second reaction unit configured to carry out a process of reacting the sample with another reagent in the container, and
a detection unit configured to carry out a process of detecting a prescribed component in a measurement specimen in the container prepared from the sample and the reagents,
the sample analysis device further comprises a control unit configured to control the sample dispensing unit and the reagent dispensing unit to carry out the process of dispensing the sample into the container and the process of dispensing the one reagent into the container and to carry out the process of dispensing the another reagent into the container after the reaction process of the sample and the one reagent is carried out,
the container transfer portion is configured to transfer the container, into which the one reagent and the another reagent have been dispensed by the reagent dispensing unit, to the second layer, and
the detection unit is configured to carry out a process of detecting the prescribed component in the measurement specimen in the container prepared by reaction in the second reaction unit.

9. The sample analysis device according to claim 8, wherein
the sample is a blood specimen,
the one reagent contains a capturing antibody for capturing an antigen in the blood specimen and magnetic particles bound to the capturing antibody,
the another reagent contains an enzyme bound to the antigen in the blood specimen and a substrate that reacts with the enzyme,
the first reaction unit is an antigen-antibody reaction unit for causing antigen-antibody reaction between the antigen and the capturing antibody in the container,
the first sample processing portion further includes a separation processing unit configured to carry out a process of separating a composite of the antigen, the capturing antibody and the magnetic particles from a reaction specimen after the antigen-antibody reaction in the container, and
the second reaction unit is an enzyme reaction unit for causing enzyme reaction between the enzyme and the substrate in the container.

10. The sample analysis device according to claim 1, wherein
the first sample processing portion includes a reagent dispensing unit configured to carry out a process of dispensing a reagent into the container, and
the reagent dispensing unit is configured to dispense the reagent into the container retained by the container transfer portion.

11. The sample analysis device according to claim 1, wherein
the second sample processing portion includes a detection unit configured to carry out a process of detecting a prescribed component in a measurement specimen in the container prepared from the sample and a reagent,
the detection unit is an optical detection unit configured to detect light emitted from the measurement specimen, and
the second layer is provided under the first layer.

12. The sample analysis device according to claim 11, wherein
the first layer is so configured that light is transmitted from outside to inside, and
the second layer is so configured that light from outside to inside is blocked.

13. The sample analysis device according to claim 1, further comprising a third sample processing portion that is arranged in a third layer positioned above or under the second layer and that is configured to carry out one part of the plurality of processes, wherein
the container transfer portion transfers the container from the second layer to the third layer.

14. The sample analysis device according to claim 1, further comprising a lower set layer arranged under the first layer and the second layer, wherein
the lower set layer includes a set region for setting a liquid container storing a liquid used for analyzing the sample.

15. The sample analysis device according to claim 1, wherein
the container transfer portion includes a container retention portion configured to retain the container and a raising/lowering mechanism configured to transfer the container from the first layer to the second layer by vertically raising/lowering the container retention portion.

16. A sample analysis device that analyzes a sample by carrying out a plurality of processes on the sample in a container, comprising:
a first base;
a first sample processing portion that is arranged on the first base and that is configured to carry out one part of the plurality of processes on the sample in the container;
a second base arranged above or under the first base; and
a second sample processing portion that is arranged on the second base and that is configured to carry out at least another part of the plurality of processes on the sample in the container, the one part of the plurality of processes having been carried out on the sample in the container; and
a container transfer portion configured to transfer the container, which contains the sample on which the one part of the plurality of processes has been carried out, from the first sample processing portion to the second sample processing portion.

17. The sample analysis device according to claim 16, wherein
the first sample processing portion includes:
a sample dispensing unit configured to carry out a process of dispensing the sample into the container, and
a reagent dispensing unit configured to carry out a process of dispensing a reagent into the container, and
the second sample processing portion includes no dispensing unit configured to carry out a process of dispensing the sample or the reagent into the container.

18. The sample analysis device according to claim 16, wherein
the first sample processing portion includes a reagent dispensing unit configured to carry out a process of dispensing a reagent into the container, and
the reagent dispensing unit is configured to dispense the reagent into the container retained by the container transfer portion.

19. The sample analysis device according to claim 16, wherein
the second sample processing portion includes a detection unit configured to carry out a process of detecting a prescribed component in a measurement specimen in the container prepared from the sample and a reagent,
the detection unit is an optical detection unit configured to detect light emitted from the measurement specimen, and
the second base is provided under the first base.

20. The sample analysis device according to claim 16, wherein
the container transfer portion includes a container retention portion configured to retain the container and a raising/lowering mechanism configured to transfer the container from the first processing portion to the second processing portion by vertically raising/lowering the container retention portion.
